(19) United States
(12) Patent Application Publication
(10) Pub. No.: US 2011/0257594 A1
(43) Pub. Date: Oct. 20, 2011

(54) NEEDLE GUIDE

(76) Inventors: Luc Lacoursiere, Quebec (CA); Daniel Gaboury, St-Augustin-de-Desmaures (CA); Louis-Alexandre Dube, Quebec (CA)

(21) Appl. No.: 12/998,993

(22) PCT Filed: Dec. 22, 2009
(86) PCT No.: PCT/CA2009/001904

§ 371 Date: Jun. 22, 2011

Related U.S. Application Data
(60) Provisional application No. 61/193,745, filed on Dec. 22, 2008.

(51) Int. Cl. A61M 5/46

(52) U.S. Cl. 604/117

(57) ABSTRACT

A needle guide (10) for guiding the insertion of a needle (12) in a patient (14), the needle guide (10) comprising: a base element (16), the base element (16) including an abutment section (20) for abutting against the patient (14) and a spacer (22) extending from the abutment section (20); and a guiding element (18), the guiding element (18) defining at least one guiding aperture (36) extending therethrough for inserting the needle (12) therethrough; the guiding element (36) being removably attachable to the spacer (22) and movable between a distal position and a proximal position. In the distal position, the guiding element (18) is attached to the spacer (22) in a spaced apart relationship relatively to the abutment section (20), and, in the proximal position, the guiding element (18) is detached from the spacer (22) and substantially adjacent to the base element (18).

### FIELD OF THE INVENTION

The present invention relates to the general field of medical procedures, and is particularly concerned with a needle guide used for percutaneous needle insertion.

### BACKGROUND

There are many medical procedures during which a needle must be relatively precisely positioned below the surface of the skin of a patient. For example, this is the case when a biopsy of a specific region of the body needs to be taken, for example in the case in which it is suspected that a mass of tissue may be cancerous. In these cases, a needle is inserted into the mass of tissue and a small sample is withdrawn for further analysis.

In other procedures, it may be necessary to position the needle precisely for many other reasons such as, for example, to precisely inject a substance for treatment of a disease in the body of a patient, to drain an abscess or to perform ablative therapies, among other procedures.

Currently, these procedures are typically performed by a physician who uses images obtained from medical imaging technologies to insert the needle through the skin and to position the needle to the best of his knowledge inside the body of the patient. However, this procedure is relatively dependent on the skills of the operator and can result in erroneous positioning, with all associated consequences regarding the treatment and diagnosis of the patient.

To alleviate these problems, in U.S. Patent Application Publication No. US2008/0146963, published on Jun. 19, 2008, Crocker et al. described a biopsy needle guide. This guide is formed by two bands of material through which apertures extend, one of the bands creating a base and the other band being arced over the base. Due to its structure, the proposed biopsy needle guide described in this document does not allow insertion of the biopsy needle to its maximal depth in the skin as the biopsy needle guide spaces apart the proximal end of the needle from the skin. Also, once applied to the patient, there is little flexibility in positioning the needle in this proposed biopsy needle guide and adjustments would have to be made by removing the biopsy needle guide from the patient and re-applying it, which can be a relatively time-consuming process. Furthermore, since the biopsy needle is only inserted in both bands in discretely positioned locations corresponding to apertures, the biopsy needle can only be inserted into the patient at discrete orientations and positions, which reduces the flexibility of this device in positioning and orienting the biopsy needle.

Against this background, there exists a need in the industry to provide an improved needle guide. An object of the present invention is therefore to provide such a needle guide.

### SUMMARY OF THE INVENTION

In a broad aspect, the invention provides a needle guide for guiding the insertion of a needle in a patient, the needle guide comprising: a base element, the base element including an abutment section for abutting against the patient and a spacer extending from the abutment section; and a guiding element, the guiding element defining at least one guiding aperture extending therethrough for inserting the needle therethrough; the guiding element being removably attachable to the spacer and movable between a distal position and a proximal position. In the distal position, the guiding element is attached to the spacer in a spaced apart relationship relatively to the abutment section, and, in the proximal position, the guiding element is detached from the spacer and substantially adjacent to the base element.

Advantageously, moving the guiding element between the proximal and distal positions takes advantages of the freedom provided for movements of the needle relative to the patient when the needle is inserted in the guiding aperture with the guiding element in the distal position and the possibility for deeper needle insertion when the guiding element is in the proximal position.

In some embodiments of the invention, the proposed needle guide is relatively small and, therefore, can be positioned at almost any location on the body of a patient, including for example the arms, the hands and the face, as well as on larger regions such as the trunk and the legs. Furthermore, the relatively small size of the device allows for its use in relatively confined spaces such as, for example, in CT scanners and MRI scanners.

In some embodiments of the invention, the abutment section is provided with an adhesive for adhesively attaching the needle guide to the skin of the patient without requiring any other device to maintain the proposed needle guide in position.

In some embodiments of the invention, the base element is provided with a relatively large base aperture through which the needle is inserted in use, which allows fine positioning of the needle without requiring moving the needle guide relatively to the patient. Also, the relatively large dimensions of the base aperture allows for positioning the needle guide roughly relatively to the patient and, afterwards, using imaging techniques to guide positioning of the needle, and move the needle over a relatively large area to precisely position the needle.

In some embodiments of the invention, the spacers create a relatively large unobstructed volume between the base element and the guiding element, which allows for easily visualizing the position of the needle relatively to the base aperture and to the patient.

In some embodiments of the invention, an array of guiding apertures extends through the guiding element, thereby allowing for many positions and orientations for the needle relatively to the needle guide and relatively to the patient. In some embodiments of the invention, the diameter of the guiding apertures increases when going away from the center of the guiding element, thereby allowing an increased range in angular positioning of the needle relatively to the needle guide when the needle is inserted through these peripheral guiding apertures. Furthermore, the presence of a multiplicity of guiding apertures allows for the insertion of many biopsy needles through the guiding element.

In some embodiments of the invention, the guiding element is positionable alternatively in both a spaced apart relationship relatively to the abutment section and in an abutment relationship relatively to the abutment section, thereby allowing a deeper insertion of the needle into the patient once positioning has been achieved.

In another broad aspect, the invention provides a method for inserting a needle in a patient using a needle guide, the needle guide comprising a base element and a guiding element defining a guiding aperture, the method comprising: securing the base element to the patient; attaching the guiding element to the base element with the guiding element in a substantially spaced apart relationship relatively to the patient; inserting the needle through the guiding aperture and into the patient at a first needle position; detaching the guiding element from the base element and moving the guiding element toward the patient; after moving the guiding element toward the patient, inserting the needle into the patient at a second needle position, the second needle position differing from the first needle position. For example, the second needle position is deeper than the first needle position. In another example, in the first and second needle positions, the needle and the patient are in different angular positional relationships relative to each other.

Advantageously, moving the guiding element toward the patient allows for reaching deeper regions in the patient. Also, this allows for reducing the angle of the needle with respect to the patient to reach otherwise unreachable portions of the patient.

In another broad aspect, the invention provides a needle guide for guiding the insertion of a needle in a patient, the needle guide comprising: a base element attachable to the patient; and a guiding element, the guiding element defining at least one guiding aperture extending therethrough for inserting the needle therethrough. The guiding element is movable between a distal position and a proximal position, wherein, in the distal position, the guiding element is attached to the base element in a spaced apart relationship relatively to the base element, and, in the proximal position, the guiding element is closer to the patient than in the distal position

In another broad aspect, the invention provides a needle guide for guiding the insertion of a needle in a patient, the needle guide comprising: a base element, the base element including an abutment section for abutting against the patient and a spacer extending from the abutment section; and a guiding element, the guiding element defining at least one guiding aperture extending therethrough for inserting the needle therethrough; the guiding element being removably attachable to the spacer in at least two different relative positional relationships between the base and guiding elements.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

FIG. 1, in a side cross-sectional view, illustrates a needle guide in accordance with an embodiment of the invention, the needle guide being shown positioned on a patient and with a needle inserted therethrough toward a mass of tissue to sample;

FIG. 2, in a perspective view, illustrates the needle guide shown in FIG. 1 with the needle also shown in FIG. 1, the needle guide being shown in a first configuration;

FIG. 3, in a perspective view, illustrates the needle guide and needle shown in FIGS. 1 and 2, with the needle guide in a second configuration;

FIG. 4, in an alternative perspective view, illustrates the needle guide shown in FIGS. 1 to 3 in the first configuration; and

FIG. 5, in a perspective view, illustrates a needle guide in accordance with an alternative embodiment of the present invention.

### DETAILED DESCRIPTION

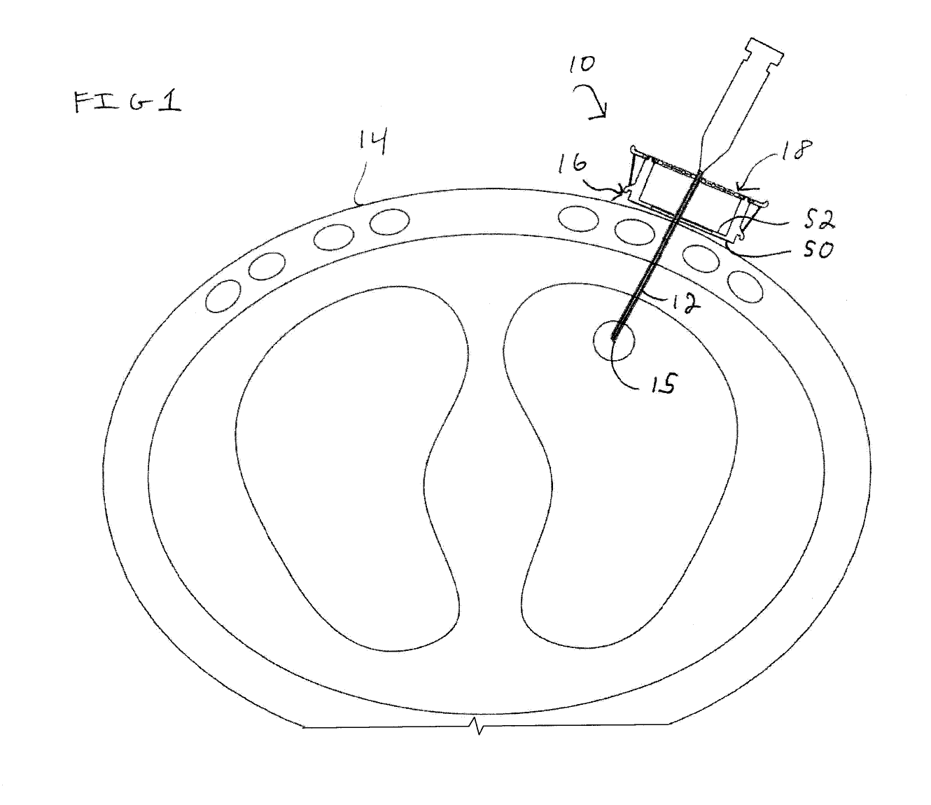

Referring to FIG. 1, there is shown a needle guide 10 in accordance with an embodiment of the present invention. The needle guide 10 is usable for guiding the insertion of a needle 12 in a patient 14. Typically, the needle 12 needs to be inserted in a patient 14 so as to be positioned at a relatively precise location 15 in the patient 14.

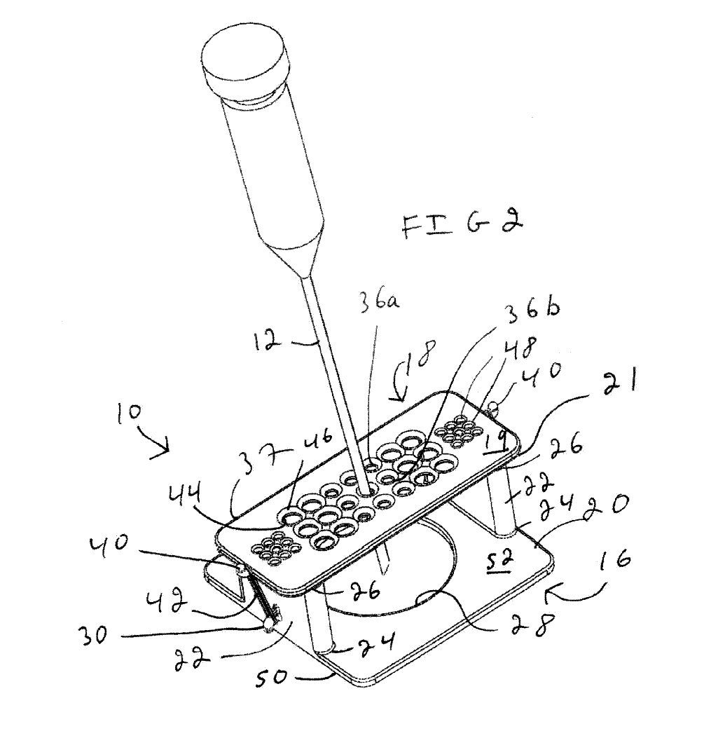

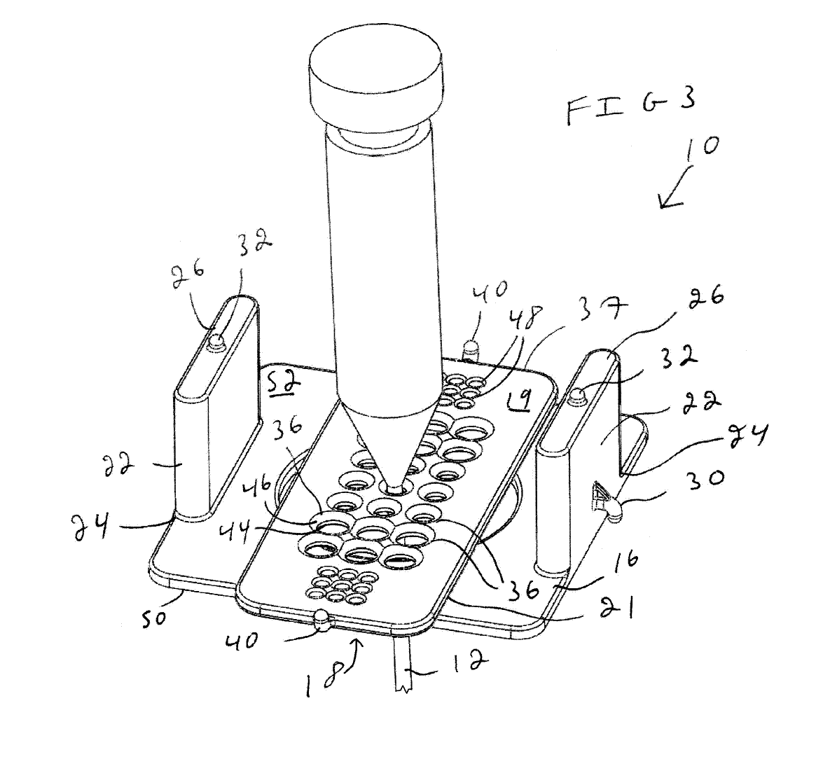

The needle guide 10 includes a base element 16 and a guiding element 18. The guiding element 18 is removably attachable to the base element 16 and is positionable in a first configuration relatively to the base element 16, as seen in FIG. 2, and a second configuration relatively to the base element 16, as seen in FIG. 3. In the second configuration, the guiding element 18 is positioned closer to the patient 14 than in the first configuration.

Referring to FIG. 2, the base element 16 includes an abutment section 20 for abutting against the patient 14 and a spacer 22 extending from the abutment section 20. In the embodiment of the invention shown in the drawings, the base element 16 includes a pair of substantially opposed spacers 22 extending from the abutment section 20. For example, the spacers 22 are substantially perpendicular to the abutment section 20, substantially longitudinally opposed relatively to each other and located substantially adjacent to the peripheral edge of the abutment section 20. Each of the spacers 22 defines a spacer proximal end 24 and a substantially opposed spacer distal end 26, the spacer proximal end 24 being located substantially adjacent to the abutment section 20 and the spacer distal end 26 being in a spaced apart relationship relatively to the abutment section 20.

The abutment section 20 defines a base aperture 28 extending therethrough. Typically, the base aperture 28 is of relatively large dimensions and has, for example, a diameter of at least 50% of the distance between the spacers 22. Typically, the base aperture 28 is substantially centrally located on the abutment section 20 and the spacers 22 are provided substantially opposed to each other with respect to the base aperture 28.

In the embodiment of the invention shown in the drawings, the abutment section 20 is substantially plate shaped. The abutment section 20 defines an abutment section proximal surface 50 for abutting against the patient and a substantially opposed abutment section distal surface 52. The base aperture 28 extends through the abutment section 20 between the abutment section proximal and distal surfaces 50 and 52. The spacer 22 extend from the abutment section distal surface 52. However, in alternative embodiments of the invention, the abutment section 20 is of any other suitable shape.

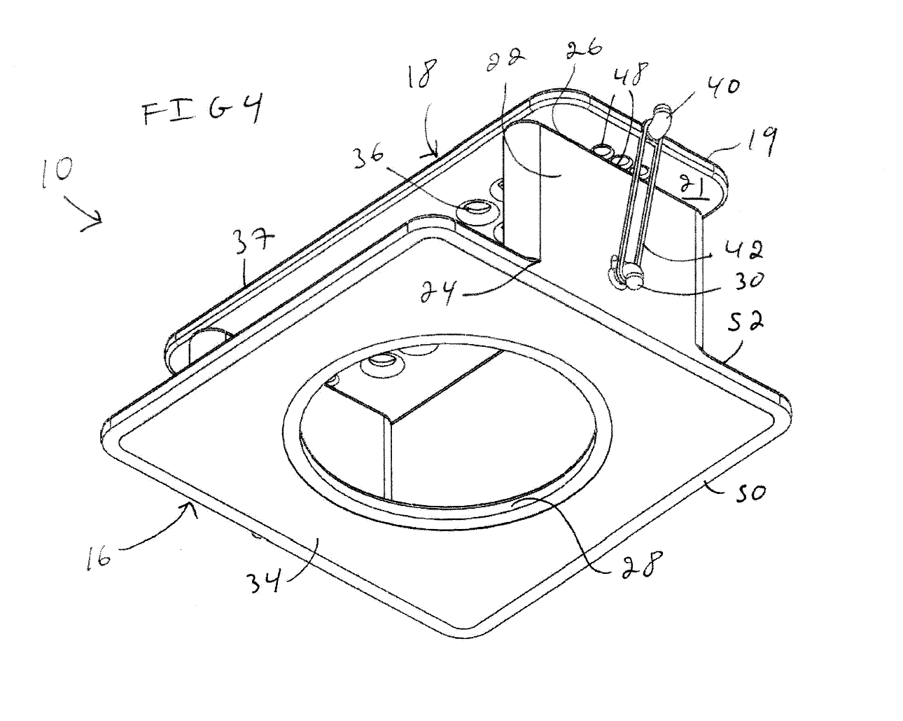

Also, in some embodiments of the invention, the abutment section 20 is provided with an adhesive 34, seen in FIG. 4, for adhesively attaching the abutment section 20 to the patient 14. The adhesive 34 is provided on the abutment section proximal surface 50.

Referring to FIG. 3, the spacers 22 are each provided with an outwardly facing attachment hook 30, the attachment hooks 30 being usable to attach the guiding element 18 to the base element 16 as described in further details hereinbelow. Also, a positioning protrusion 32 extends substantially away from the base element 16 from each of the spacers 22 at the spacer distal end 26.

The guiding element 18 is substantially plate-shaped and defines a guiding element first side 19 and a substantially opposed guiding element second side 21. The guiding element 18 is delimited by a guiding element peripheral edge 37. At least one guiding aperture 36 extends through the guiding element 18 between the guiding element first and second sides 19 and 21 for allowing insertion of the needle 12 therethrough. Typically, a plurality of guiding apertures 36 extend through the guiding element 18 between the guiding element first and second sides 19 and 21 each for allowing selective insertion of the needle 12 therethrough Typically, the base aperture 28 is of larger diameter than the guiding apertures 36.

The guiding element 18 is removably attachable to the base element 16 so as to be positionable in the first and second configurations. More specifically, the guiding element 18 is removably attachable to the spacers 22 and movable between a distal position and a proximal position. In the distal position, the guiding element 18 is attached to the spacers 22 in a spaced apart relationship relatively to the abutment section, which corresponds to the first configuration. In the proximal position, the guiding element 18 is detached from the spacers 22 and substantially adjacent to the base element 16, which corresponds to the second configuration. In some embodiments of the invention, the guiding element 18 is located between the spacers 22 in the proximal position.

In some embodiments of the invention, at least one of the guiding apertures 36 and the base aperture 28 are substantially in register with each other when the guiding element 18 is in the proximal position, in the distal position, or both in the proximal and distal positions. In other embodiments of the invention, the needle guide 10 is configured and sized so that the needle 12 is insertable simultaneously through both the guiding and base apertures 36 and 28 when the guiding element 18 is in the distal and proximal positions. In a more specific embodiment of the invention, the needle guide 10 is configured and sized so that the guiding element 18 is movable between the proximal and distal positions with the needle 12 inserted simultaneously through both the guiding and base apertures 36 and 28. These embodiments allow to different extents the configuration of the needle guide 10 between the first and second configurations with the needle 12 inserted in the patient to profit from the advantages provided by these two configurations in a single procedure, and in some of these embodiments, without withdrawing the needle 12 from the patient 14 when the guiding element 18 is moved.

In some embodiments of the invention, a plurality of guiding apertures 36 is provided, each guiding aperture 36 extending through the guiding element 18. In some embodiments of the invention, the guiding apertures 36 are arranged in a substantially grid-like configuration. This configuration facilitates location of a suitable guiding aperture 36 through which the needle 12 needs to be inserted to reach a given location 15. However, in alternative embodiments of the invention, the guiding aperture 36 are arranged in any other suitable configuration.

In some embodiments of the invention, all the guiding apertures 36 have similar configurations and dimensions. In other embodiments of the invention, the guiding apertures 36 located near the centre of the guiding element 18 are of a substantially smaller diameter than the guiding apertures 36 located further away from the centre of the guiding element 18. More specifically, as seen in FIG. 2, the guiding apertures 36 include a first guiding aperture, for example guiding aperture 36a, having a first diameter, and a second guiding aperture, for example guiding aperture 36b, having a second diameter. The first guiding 36a aperture is closer to the guiding element peripheral edge 37 than the second guiding aperture 36b and the first diameter is larger than the second diameter. This configuration allows for positioning the needle 12 at different angles relatively to the guiding element 18, and more specifically to have larger angles relatively to the patient when inserting the needle 12 in guiding apertures 36 located further away from the centre of the guiding element 18, which allows access to subcutaneous structures substantially in register with the centre of the base aperture 28.

In some embodiments of the invention, as better seen in FIG. 3, each of the guiding apertures 36 includes a substantially cylindrical portion 44 located substantially centrally in the guiding aperture 36 and peripherally located substantially frusto-conical portions 46. In other words, the frusto-conical portions 46 are located substantially adjacent one of the guiding element first and second sides 19 and 21 and the cylindrical portion 44 extends therebetween. The frusto-conical portions 46 each taper in a direction leading toward the cylindrical portion 44. However, in alternative embodiments of the invention, the guiding apertures 36 have any other suitable shape, such as a substantially cylindrical shape, among other possibilities.

The guiding element 18 includes a pair of substantially opposed attachment hooks 40 that are each positionable substantially adjacent to one of the attachment hooks 30 of the base element 16 when the guiding element 18 is in the first configuration. Biasing elements 42, for example in the form of rubber bands, seen in FIG. 2, are removably attachable to both the attachment hooks 30 and the attachment hooks 40 so as to bias the guiding element 18 toward the base element 16 when the guiding element 18 is positioned in the first configuration. The attachment hooks 30 and 40 and the biasing element 42 together form a lock 31 for reversibly locking the guiding element 18 and each spacer 22 to each other when the guiding element 18 and the spacers 22 are attached to each other with the guiding element 18 in the distal position.

Typically, the guiding element 18 also defines two arrays of positioning apertures 48 extending therethrough between the guiding element first and second sides 19 and 21 each provided substantially adjacent a respective one of the attachment hooks 40. The positioning apertures 48 are each provided for receiving one of the positioning protrusions 32. By inserting the positioning protrusions 32 in a different positioning aperture 48, the position of the guiding element 18 relatively to the base element 16 can be finely tuned. In some embodiments of the invention, this fine tuning of the position of the guiding element 18 can be performed with a needle 12 extending through one of the guiding apertures 36 and partially inserted in the patient 14, for example to adjust the penetration angle of the needle 12. In other words, the guiding element 18 is removably attachable to the spacers 22 in at least two different relative positional relationships between the base and guiding elements 16 and 18. Typically, the guiding element 18 remains substantially coplanar with itself when moved between the at least two different relative positional relationships. The positioning apertures 48 cause the at least two different relative positional relationships between the base and guiding elements 16 and 18 to be spatially spaced apart by discrete distance increments. While the positioning apertures 48 extend through the guiding element 18, it is within the scope of the invention to replace the positioning apertures 48 by recesses that extend only partially into the guiding element 18.

In use, the abutment section 20 of the base element 16 is positioned over the patient 14 at a desired location and secured to the patient 14 using the adhesive 34. Then, the guiding element 18 is attached in the first configuration to the base element 16 and positioned to a desired location relative to the abutment section 20 using the positioning protrusions 32 and the positioning apertures 48. Afterwards, if desired, the biasing elements 42 are attached to adjacent attachment hooks 30 and 40 to secure the guiding element 18 and the base element 16 to each other. Then, the needle 12 is inserted through one the guiding apertures 36, through the base aperture 28 and inserted at the desired location 15 in the patient 14. In some embodiments of the invention, if the guiding element 18 is found not to be properly positioned with respect to the patient 14, the guiding element 18 and the base element 16 are detached from each other and reattached to each other so as to change a relative positional relationship between the base and guiding elements 16 and 18 by inserting the positioning protrusion 32 in a different positioning aperture 48.

If the site at which the biopsy needs to be taken is located relatively deep in the patient 14 and cannot be reached with the guiding element 18 in the first configuration, the guiding element 18 is detached from the base element 16 by removing the biasing elements 42 from the attachment hooks 30 and 40, turning the guiding element 18 at about 90 degrees relatively to the base element 16 and configuring the guiding element 18 in the second configuration, which moves the guiding element 18 toward the patient 14. This configuration allows for deeper penetration of the needle 12 inside the patient 14. Since the needle 12 has been inserted over a relatively large distance in the patient 14 before this operation is performed, orientation of the needle 12 relatively to the patient 14 remains relatively fixed during this operation and, therefore, causes little or no imprecision in the positioning of the needle 12. Afterwards, the needle 12 is removed from the patient after performing the intended procedure.

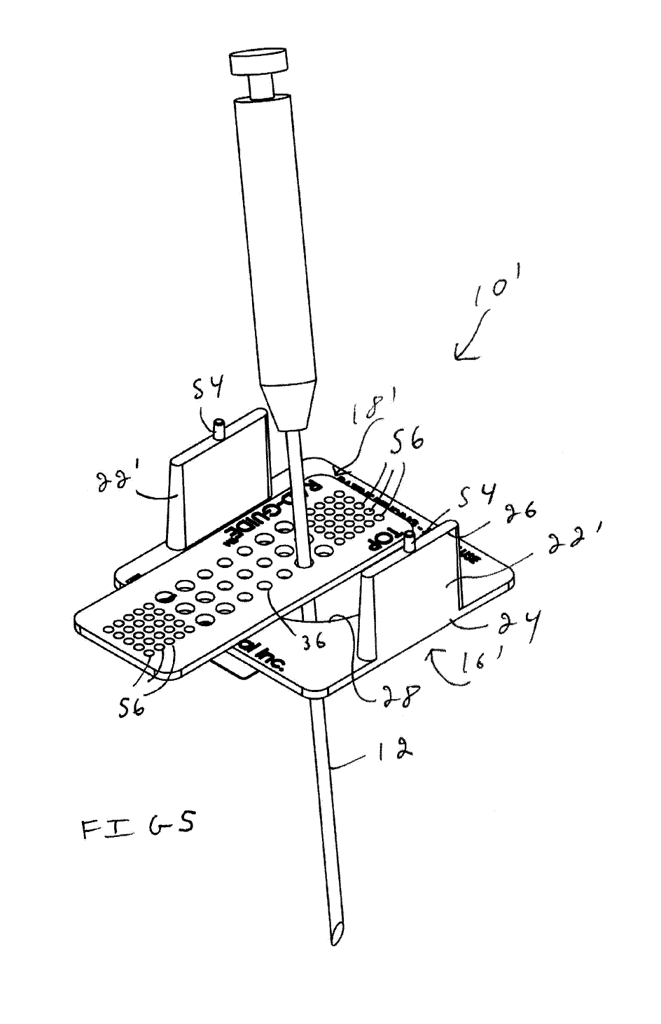

FIG. 5 illustrates a needle guide 10′ in accordance with an alternative embodiments of the present invention. In the needle guide 10′, the guiding element 18 and the spacer 22 are attachable to each other in a press-fit relationship relatively to each other. This embodiment therefore does not require that the biasing elements 42 present in the needle guide 10 be present. Otherwise, the needle guides 10 and 10′ are substantially similar.

To that effect, the spacer 22 defines a pin 54 protruding therefrom and the guiding element 18′ defines at least one, and typically a plurality of attachment hollows 56 for selectively and alternatively receiving the pin 54 when the spacer 22 and guiding element 18′ are attached to each other. The attachment hollows 56 extends through the guiding element 18′ and the pin 54 typically protrudes from the attachment hollow 56 when the guiding element 18 is attached to the spacer 22. In some embodiments of the invention, the pin 54 and the hollow 56 both have a substantially frusto-conical configuration tapering in a direction leading substantially away from the base elements 16. This configuration facilitates secure attachment of the guiding element 18′ and the base element 16 to each other as pressing these two elements 18′ and 16 toward each other increases the friction therebetween and locks these two elements to 18′ and 16 to each other.

In some embodiments of the invention, the material used for manufacturing the needle guide 10 is plastic. This material is relatively light and relatively rigid and, therefore, can be used to maintain in a desired orientation and position relatively large and heavy needles 12 and associated devices. Typically, the proposed material is relatively translucent to X-rays and, therefore, does not impede X-ray imaging of the region of interest through which the needle 12 should be inserted. Furthermore, the material is sufficiently radio-opaque for being seen on CT imaging, allowing precise location of each guiding aperture 36 through which the needle 12 will be inserted. This allows choosing the exact pathway of the needle 12 from the patient's skin to the desired location 15. This material is typically not sufficiently opaque to cause CT artefacts. Also, the proposed material is easily sterilizable using known methods and devices.

Although the present invention has been described hereinabove by way of preferred embodiments thereof, it can be modified, without departing from the spirit and nature of the subject invention as defined in the appended claims.

1. A needle guide for guiding the insertion of a needle in a patient, said needle guide comprising:
a base element, said base element including an abutment section for abutting against said patient and a spacer extending from said abutment section; and
a guiding element, said guiding element defining at least one guiding aperture extending therethrough for inserting said needle therethrough;
said guiding element being removably attachable to said spacer and movable between a distal position and a proximal position, wherein, in said distal position, said guiding element is attached to said spacer in a spaced apart relationship relatively to said abutment section, and, in said proximal position, said guiding element is detached from said spacer and substantially adjacent to said base element.

2. A needle guide as defined in claim 1, wherein said abutment section defines a base aperture extending therethrough, said guiding aperture and said base aperture being substantially in register with each other when said guiding element is in said proximal position.

3. A needle guide as defined in claim 2, wherein said guiding aperture and said base aperture are substantially in register with each other when said guiding element is in said distal position.

4. A needle guide as defined in claim 2, wherein said base aperture has a larger diameter than said guiding aperture.

5. A needle guide defined in claim 2, wherein said needle guide is configured and sized so that said needle is insertable simultaneously through both of said guiding and base apertures when said guiding element is in said distal and proximal positions.

6. A needle guide defined in claim 5, wherein said needle guide is configured and sized so that said guiding element is movable between said proximal and distal positions with said needle inserted simultaneously through both said guiding and base apertures.

7. A needle guide as defined in claim 1, wherein said guiding element defines a plurality of guiding aperture extending therethrough each for selectively inserting said needle therethrough.

8. A needle guide as defined in claim 7, wherein said guiding element defines a guiding element peripheral edge, said plurality of guiding apertures including a first guiding aperture having a first diameter and a second guiding aperture having a second diameter, said first guiding aperture being closer to said guiding element peripheral edge than said second guiding aperture and said first diameter being larger than said second diameter.

9. A needle guide as defined in claim 1, wherein said guiding element is substantially plate-shaped.

10. A needle guide as defined in claim 1, wherein said guiding element and said spacer are attachable to each other in a press-fit relationship relatively to each other.

11. A needle guide as defined in claim 1, wherein said spacer defines a pin protruding therefrom and said guiding element defines an attachment hollow for receiving said pin when said spacer and guiding element are attached to each other.

12. A needle guide as defined in claim 11, wherein said attachment hollow extends through said guiding element and said pin protrudes from said attachment hollow when said guiding element is attached to said spacer.

13. A needle guide as defined in claim 1, wherein said spacer defines a pin protruding therefrom and said guiding element defines at least two attachment hollows each for selectively and alternatively receiving said pin when said spacer and guiding elements are attached to each other.

14. A needle guide as defined in claim 1, wherein said abutment section is substantially plate-shaped.

15. A needle guide as defined in claim 1, wherein said guiding aperture defines a substantially frusto-conical portion.

16. A needle guide as defined in claim 1, wherein said abutment section is substantially plate-shaped and defines an abutment section proximal surface for abutting against said patient and a substantially opposed abutment section distal surface, said abutment section also defining a base aperture extending therethrough between said abutment section proximal and distal surfaces.

17. A needle guide as defined in claim 16, further comprising another spacer, both of said spacers extending from said abutment section distal surface.

18. A needle guide as defined in claim 17, wherein said spacers are provided substantially opposed to each other with respect to said base aperture.

19. A needle guide as defined in claim 18, wherein said guiding element is located between said spacers in said proximal position.

20. A needle guide as defined in claim 19, wherein said guiding element is substantially plate-shaped.

21. A needle guide as defined in claim 1, wherein said abutment section is provided with an adhesive for adhesively attaching said abutment section to said patient.

22. A needle guide as defined in claim 1, further comprising a lock for reversibly locking said guiding element and said spacer to each other when said guiding element and said spacer are attached to each other with said guiding element in said distal position.

23. A needle guide as defined in claim 1, wherein said guiding element defines a plurality of guiding apertures extending therethrough each for selectively inserting said needle therethrough, said guiding apertures being arranged in substantially grid-like configuration.

24. (canceled)

25. (canceled)

26. (canceled)

27. (canceled)

28. (canceled)

29. (canceled)

30. A needle guide for guiding the insertion of a needle in a patient, said needle guide comprising:
a base element attachable to said patient; and
a guiding element, said guiding element defining at least one guiding aperture extending therethrough for inserting said needle therethrough;
said guiding element being movable between a distal position and a proximal position, wherein, in said distal position, said guiding element is attached to said base element in a spaced apart relationship relatively to said base element, and, in said proximal position, said guiding element is closer to said patient than in said distal position.

31. A needle guide for guiding the insertion of a needle in a patient, said needle guide comprising:
a base element, said base element including an abutment section for abutting against said patient and a spacer extending from said abutment section; and
a guiding element, said guiding element defining at least one guiding aperture extending therethrough for inserting said needle therethrough;
said guiding element being removably attachable to said spacer in at least two different relative positional relationships between said base and guiding elements.

32. A needle guide as defined in claim 31,
